(19) United States
(12) Patent Application Publication
(10) Pub. No.: US 2016/0120524 A1
(43) Pub. Date: May 5, 2016

(54) APPARATUS AND METHOD FOR SUPPORTING BIOPSY

(71) Applicant: CANON KABUSHIKI KAISHA, Tokyo (JP)

(72) Inventor: Nobuhito Suehira, Tokyo (JP)

(21) Appl. No.: 14/896,183

(22) PCT Filed: Jun. 12, 2014
(86) PCT No.: PCT/JP2014/003138

§ 371 Date: Dec. 4, 2015

(30) Foreign Application Priority Data
Jun. 13, 2013 (JP) 2013-125069
Jun. 13, 2013 (JP) 2013-125070
Jun. 13, 2013 (JP) 2013-125071

(51) Int. Cl. A61B 10/04; A61B 34/30; A61B 10/02; A61B 8/08; A61B 8/00

(52) U.S. Cl. CPC A61B 10/04; A61B 8/0841; A61B 8/085; A61B 8/5207; A61B 8/4416; A61B 10/0275; A61B 34/30; A61B 8/0825; A61B 8/54; A61B 2017/3413

(57) ABSTRACT

The present invention relates to a biopsy support apparatus. The apparatus includes an ultrasonic unit configured to generate an ultrasonic image on the basis of a signal from an ultrasonic probe in contact with a living organism; an optical-image acquisition unit configured to generate an optical image on the basis of a signal from a biopsy probe inserted in the living organism; and

a control unit configured to control the ultrasonic unit and the optical-image acquisition unit. The control unit calculates the positional relationship between the biopsy probe and the target by aligning a feature portion in the ultrasonic image and a feature portion corresponding thereto in the optical image and controls the puncture state of the biopsy probe on the basis of the positional relationship.

### TECHNICAL FIELD

The present invention relates to an apparatus and a method for supporting biopsy by puncture.

### BACKGROUND ART

A mammography apparatus and an ultrasonic apparatus are used for breast cancer screening. Determination of whether a detected lesion is benign or malignant is performed by biopsy and pathological diagnosis. The image resolution of ultrasonic-guided biopsy is low, thus making it difficult to determine whether desired tissue has entered the puncture needle. Therefore, the success rate is increased by, for example, increasing the number of times of biopsy or increasing the amount of tissue collected per biopsy. PTL 1 discloses a puncture support system for automatically bringing a puncture needle to a target by a simple operation under ultrasonic guide. This system associates the position and orientation of an ultrasonic probe with an ultrasonic image obtained with the probe to obtain the three-dimensional position of a target in target tissue and controls the puncture needle so that it reaches the position.

PTL 2 discloses an endoscope using an optical coherence tomography unit (hereinafter referred to as an OCT unit). This allows tissue to be cut off with a biopsy forceps after biological tissue is detected by the OCT unit. However, an endoscope to be inserted into a lumen, such as a digestive organ, is about 10 mm in diameter, and thus a plurality of ports, such as a biopsy forceps port, an OCT observation port, and a normal observation ports, can be disposed away from each other. In contrast, puncture cytology uses a needle having an outside diameter of 1 mm or less, and core needle biopsy uses a needle having an outside diameter of about 1 mm to 2 mm, which are thinner than that of endoscopes for use in the field of digestive organs. Breast biopsy requires a puncture needle having the smallest possible outside diameter to decrease the invasiveness and needing a minimum number of times of biopsy, thereby increasing the success rate.

Mammography allows a minute lesion, typified by microcalcification, to be detected. However, mammography involves exposure to radiation and thus cannot be frequently used. On the other hand, microcalcification cannot be observed by an ultrasonic apparatus and an MR apparatus, and thus, puncture under such modalities is not performed. PTL 3 discloses a mammography treatment apparatus that specifies a seat of disease by acquiring an X-ray image of a breast with an X-ray machine, with the breast fixed with a pressure plate, and that treats the seat of disease.

### CITATION LIST

### Patent Literature

PTL 1: Japanese Patent Laid-Open No. 2012-035010

PTL 2: Japanese Patent Laid-Open No. 2002-263055

PTL 3: Japanese Patent Laid-Open No. 2006-280444

### SUMMARY OF INVENTION

### Technical Problem

However, since the resolution of ultrasonic images is not sufficient and the breast is sometimes deformed during puncture, it is difficult to check whether a set target can be correctly punctured. Another problem is that it cannot be determined whether the set target itself is correct. Therefore, increasing the success rate of biopsy results in an increase in the number of times of collection and the amount of collection per puncture, which imposes a burden on the subject.

If tissue around the puncture needle can be observed at a level close to that of a microscope to allow the presence of lesion to be reliably detected, the weak point of the ultrasonic apparatus can be complemented. The present invention provides a biopsy support apparatus configured to support ultrasonic guide biopsy while observing tissue around a puncture needle at optical-microscope-level resolution.

If microcalcified lesion is detected by mammography, and biopsy is to be performed another day after informed consent, it is difficult to specify the position of the lesion with a conventional biopsy support apparatus. Preferably, the position of the lesion during puncture can be specified on the basis of existing image data of mammography, without the breast being shaped in the same physical form at the examination and the biopsy.

The present invention provides a biopsy support system described below. The biopsy support system introduces a puncture needle into a lesion using image data obtained from the outside of a living organism by past mammography or the like and acquires an image of tissue close to the lesion by acquiring an optical image from the interior of the living organism.

### Solution to Problem

According to a first aspect of the present invention, provided is a biopsy support apparatus configured to acquire an ultrasonic image and an optical image of a living organism and to puncture a target in the living organism on the basis of the ultrasonic image and the optical image. The apparatus includes an ultrasonic unit configured to generate the ultrasonic image on the basis of a signal from an ultrasonic probe in contact with the living organism; an optical-image acquisition unit configured to generate the optical image on the basis of a signal from a biopsy probe inserted in the living organism; and a control unit configured to control the ultrasonic unit and the optical-image acquisition unit. The control unit sets the scanning direction of the ultrasonic probe and the moving direction of the biopsy probe in substantially the same plane, calculates the positional relationship between the biopsy probe and the target by aligning a feature portion in the ultrasonic image and a feature portion corresponding thereto in the optical image, and controls the puncture state of the biopsy probe on the basis of the positional relationship.

According to a second aspect of the present invention, provided is a biopsy support apparatus for collecting part of biological tissue. The apparatus includes a search needle including an optical device for acquiring an optical image of biological tissue and an exit window; and a puncture needle including a storage portion for storing the part of the biological tissue collected. The puncture needle is slidable using the search needle as a guide. Optical images are acquired in a first state in which the exit window projects from the storage portion and in a second state in which the exit window is located along the storage portion.

According to a third aspect of the present invention, provides is a biopsy support apparatus for puncturing a target in a subject. The apparatus includes an optical-image acquisition unit configured to generate an optical image of the subject by inserting a biopsy probe into the subject; and a control unit configured to control the optical-image acquisition unit and the puncture state of the biopsy probe. The control unit acquires data on a 3D image of the subject acquired by a 3D-image acquisition unit in advance; and determines the penetration path of the biopsy probe on the basis of the data on the 3D image.

Further features of the present invention will become apparent from the following description of exemplary embodiments with reference to the attached drawings.

### BRIEF DESCRIPTION OF DRAWINGS

FIG. 1 is a block diagram of a biopsy support apparatus according to a first embodiment.

FIG. 2A is an overall view of a biopsy probe according to the first embodiment.

FIG. 2B is a top view of a puncture needle according to the first embodiment and a cross-sectional view taken along line A-A′.

FIG. 2C is a side view of the puncture needle and a cross-sectional view taken along line B-B′.

FIG. 2D is a sectional view of a search needle according to the first embodiment.

FIG. 3 is a flowchart for explaining a method for supporting biopsy according to the first embodiment.

FIG. 4A is a schematic diagram for explaining the relationship among a breast, an ultrasonic probe, and the puncture needle according to the first embodiment.

FIG. 4B is a side view of the same.

FIG. 5A is a schematic diagram for explaining the relationship between an ultrasonic image and OCT images according to the first embodiment.

FIG. 5B is a schematic diagram of OCT volume data according to the first embodiment.

FIG. 6A is a schematic diagram illustrating a state in which the tissue is to be cut off according to the first embodiment.

FIG. 6B is a schematic diagram illustrating a state in which the tissue is to be cut off according to the first embodiment.

FIG. 6C is a schematic diagram illustrating a state in which the tissue is to be cut off according to the first embodiment.

FIG. 6D is a schematic diagram illustrating a state in which the tissue is to be cut off according to the first embodiment.

FIG. 7A is a schematic diagram of a puncture needle according to a second embodiment.

FIG. 7B is a schematic diagram of another puncture needle according to the second embodiment.

FIG. 7C is a schematic diagram of another puncture needle according to the second embodiment.

FIG. 7D is a schematic diagram of another puncture needle according to the second embodiment.

FIG. 8A is an explanatory diagram of an OCT image according to the second embodiment.

FIG. 8B is an explanatory diagram of volume data acquired in the moving direction according to the second embodiment.

FIG. 8C is an explanatory diagram of an OCT image according to the second embodiment.

FIG. 8D is an explanatory diagram of an observation area according to the second embodiment.

FIG. 9 is a flowchart for explaining a method for supporting biopsy according to a third embodiment.

FIG. 10A is a schematic diagram for explaining the relationship among a breast, an ultrasonic probe, and a puncture needle according to the third embodiment.

FIG. 10B is a side view of the same.

FIG. 11A is a schematic diagram of OCT volume data according to the third embodiment.

FIG. 11B is a schematic diagram of an ultrasonic image according to the third embodiment.

FIG. 11C is a schematic diagram of a mammography image according to the third embodiment.

FIG. 12A is a schematic diagram illustrating a state in which the tissue is to be cut off according to the third embodiment.

FIG. 12B is a schematic diagram illustrating a state in which the tissue is to be cut off according to the third embodiment.

FIG. 12C is a schematic diagram illustrating a state in which the tissue is to be cut off according to the third embodiment.

FIG. 12D is a schematic diagram illustrating a state in which the tissue is to be cut off according to the third embodiment.

### DESCRIPTION OF EMBODIMENTS

Embodiments of the present invention will be described in detail hereinbelow with reference to the drawings.

### First Embodiment

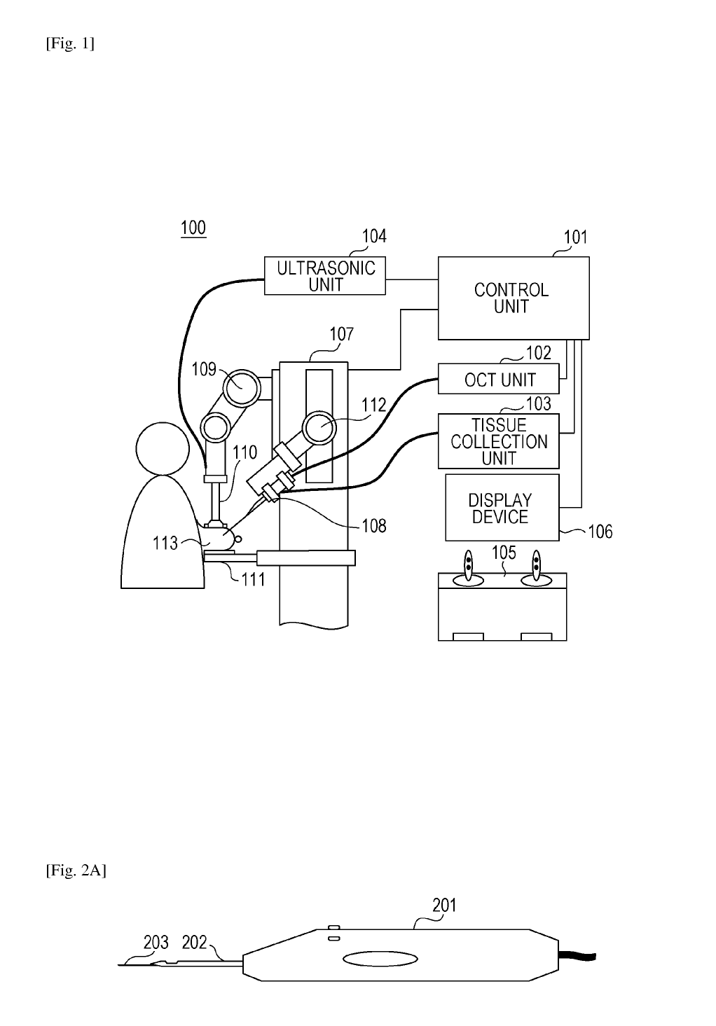

FIG. 1 is a schematic block diagram of a biopsy support apparatus 100 for breasts.

The biopsy support apparatus 100 increases the accuracy of biopsy by controlling an ultrasonic probe 110 and a biopsy probe 108 by using a robot system. A breast examining table 111, an ultrasonic probe arm 109, and a biopsy probe arm 112 are disposed on a fixed stand 107. The ultrasonic probe arm 109 controls the position of the ultrasonic probe 110, and the biopsy probe arm 112 controls the operation of the biopsy probe 108. The OCT unit 102 (corresponding to an optical-image acquisition unit) generates an optical coherence tomography image (OCT image) (corresponding to an optical image) on the basis of a signal from the biopsy probe 108. Light used has a central wavelength of 1.3 micrometers, which makes the light easily enter a living organism (subject). The depth resolution used is about 10 micrometers. The penetration depth is within 3 mm, although it depends on the tissue. An ultrasonic unit 104 generates an ultrasonic image on the basis of a signal from the ultrasonic probe 110 in contact with a living organism (subject). Ultrasonic waves used are generated from a linear probe having a frequency of 7 MHz to 15 MHz and 256 channels. The wavelength of the ultrasonic waves is about 150 micrometers if the ultrasonic waves have passed through water at a frequency of 10 MHz. Therefore, the distance resolution is about 150 micrometers at a wavenumber of 2, about 400 micrometers at a wavenumber of 5, and about 750 micrometers at a wavenumber of 10. A tissue collection unit 103 holds biological tissue collected by the biopsy probe 108. The control unit 101 cooperatively controls the OCT unit 102, the tissue collection unit 103, and the ultrasonic unit 104.

A breast 113 is placed on the breast examining table 111. An ultrasonic image of the breast can be acquired using the ultrasonic probe 110. The initial positions of the ultrasonic probe 110 and the biopsy probe 108 are roughly and manually determined by an operator. Thereafter, the operator operates the ultrasonic probe 110 and the biopsy probe 109 with a console 105 while viewing a display device 106. Examples of the console 105 include a joystick controller, a push button switch, and a foot pedal.

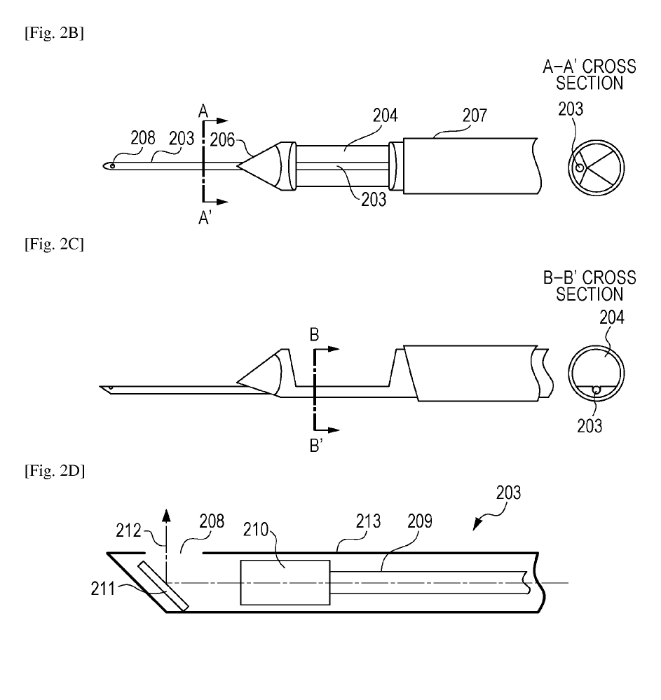

FIGS. 2A to 2D are diagrams illustrating the structure of a biopsy probe for a breast. FIG. 2A is an overall view; FIG. 2B is a top view of a puncture needle and a cross-sectional view taken along line A-A′; FIG. 2C is a side view of the puncture needle and a cross-sectional view taken along line B-B′; and FIG. 2D is a sectional view of a search needle. A puncture needle 202 extends from a biopsy-probe main body 201. A search needle 203 extends from the puncture needle 202. An operator collects part of tissue while operating various buttons, with the biopsy-probe main body 201 in hand. In some cases, a foot pedal is used for operation.

The puncture needle 202 has a storage portion 204 for storing biological tissue. The biological tissue stored in the storage portion 204 is cut off from the living organism by moving an external cylindrical cutter (cutting portion) 207 toward the storage portion 204. The outside diameter of the puncture needle 202 is, for example, about 1.2 mm to 4 mm.

The search needle 203 can be rotated 360 degrees and can be slid in a longitudinal direction from a puncture-needle distal end 206. The search needle 203 has a cylindrical metal tube 213, in which optical devices, such as an optical fiber 209, a lens 210, and a mirror 211, are disposed. A laser beam passes through the optical devices and exits outside the search needle 203 through an exit window 208. The outside diameter of the metal tube 213 is 0.4 mm to 1 mm. The search needle 203 outputs a signal for forming an optical image to the OCT unit 102. The puncture needle 202 and the search needle 203 are disposable and are replaced every time they are used.

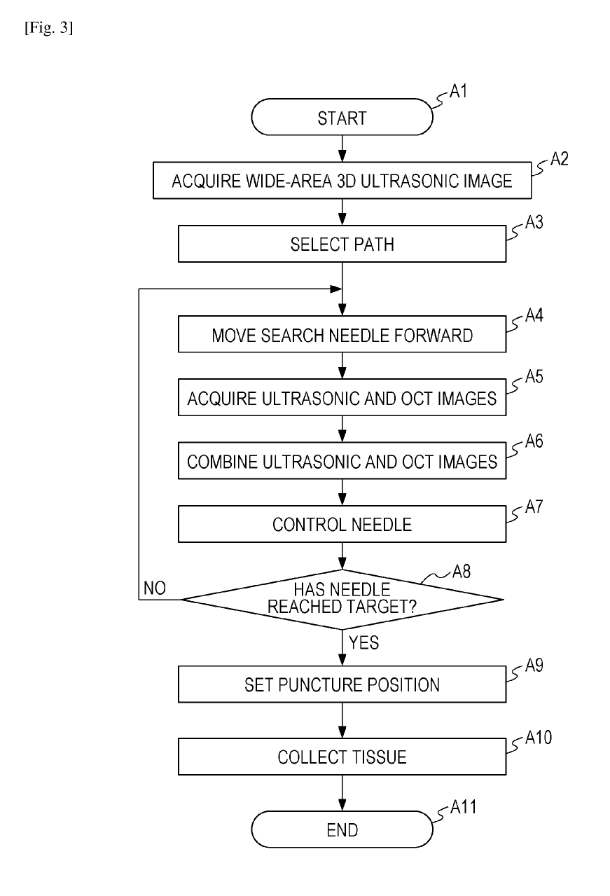

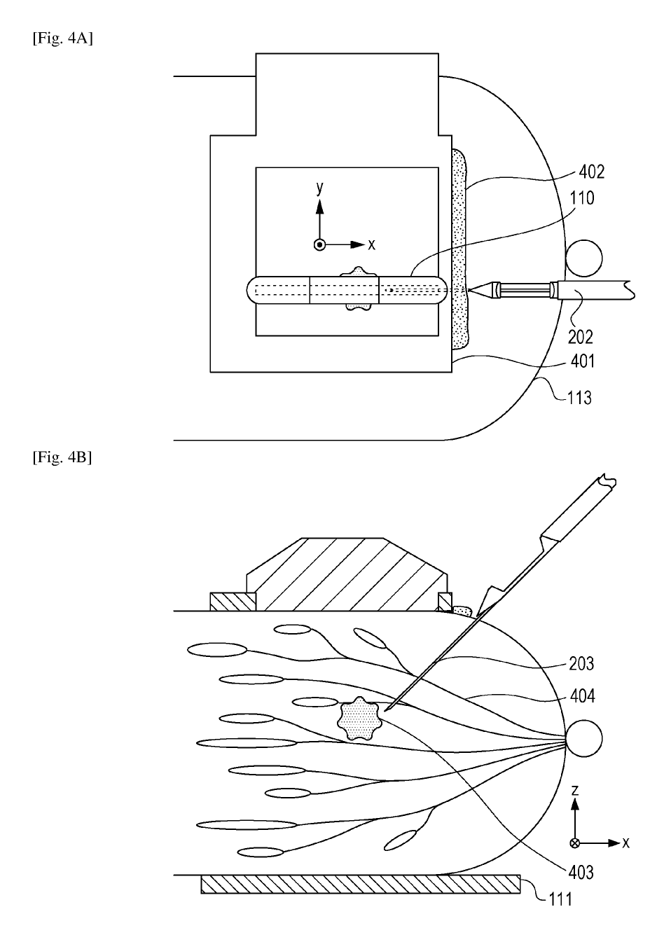

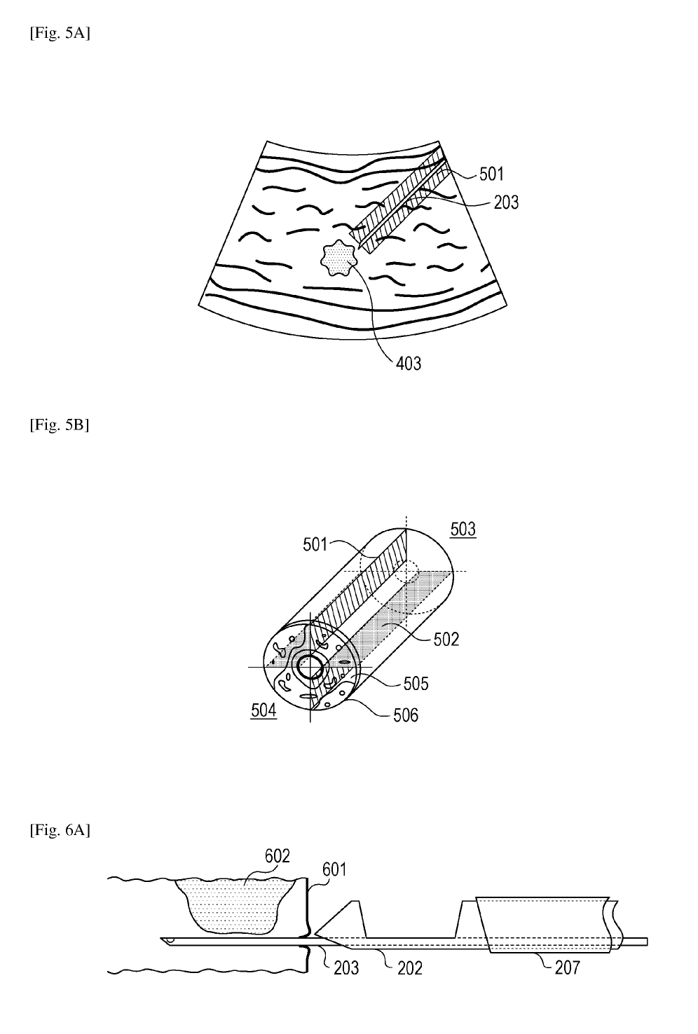

Next, a puncture process controlled by the control unit 101 will be described using a flowchart in FIG. 3. FIGS. 4A and 4B show the relationship among the breast 113, the ultrasonic probe 110, and the search needle 203. FIG. 4A is a top view, and FIG. 4B is a side view. Here, assume that a linear array of the ultrasonic probe 110 is disposed parallel to an x-y plane. FIGS. 5A and 5B show the relationship between an ultrasonic image and an OCT image. FIG. 5A illustrates an ultrasonic image, and FIG. 5B illustrates OCT volume data.

In step A1, puncture is started. The breast 113 is subjected to disinfection and anesthesia as appropriate. Although measurement is performed in a standing position, a sitting position or a supine position using a chair or a bed is possible. As shown in FIGS. 4A and 4B, the breast 113 is sandwiched between a pressure plate 401 and the breast examining table 111. Jell 402 is applied to the breast 113 for acoustic impedance matching.

In step A2, a wide-area three-dimensional (3D) ultrasonic image is acquired. As shown in FIGS. 4A and 4B, the ultrasonic probe 110 can perform one-dimensional linear electronic scanning and can acquire a 3D ultrasonic image by combining one-dimensional linear electronic scanning with one-dimensional mechanical scanning parallel to an x-y plane. In addition, mapping of a 3D blood vessel structure may be performed by Doppler imaging. Furthermore, elastic mapping of tissue may be performed in measurement using elastography or the like. Combination use of them will increase information for alignment.

In step A3, the penetration path of the search needle 203 is set. The position of the target 403 is conformed using the 3D ultrasonic image acquired in step A2. After the position of the target 403 and the penetration path of the search needle 203 are determined, the angle of the ultrasonic probe 110 is changed so that the scanning direction of the ultrasonic probe 110 and the moving direction of the biopsy probe 108 become substantially flush with each other and is fixed. These operations allow the state of penetration of the search needle 203 to be observed in real time using the ultrasonic image, as shown in FIG. 5A. The ultrasonic probe 110 may be vibrated at a small amplitude in a direction perpendicular to the search needle 203 so that the direction in which the distal end of the search needle 203 deviates from the ultrasonic probe 110 can be detected.

In step A4, the search needle 203 is inserted into the breast 113. The search needle 203 is extended from the puncture needle 202 following the path determined in step A3, and OCT imaging is performed while the search needle 203 is inserted into the tissue. Since the search needle 203 is rotated in a direction perpendicular to the longitudinal direction, a cylindrical 3D OCT image can be acquired as it moves forwards. Straight movement is controlled independently from rotation; 1 mm per second, or, for example, 0.5 mm or 2 mm, according to circumstances. For rotation, the search needle 203 is rotated at 10 revolutions per millimeter or 100 revolutions per millimeter to acquire an image. Control of straight movement and rotation can be selected as appropriate; for example, every 100 micrometers for 10 revolutions per millimeter and continuously for 100 revolutions per millimeter. Image acquisition may be increased as the search needle 203 approaches the target 403. Ultrasonic images, if having a resolution of about 400 micrometers, are aligned with OCT images acquired every 100 micrometers.

In step A5, the control unit 101 acquires ultrasonic images and OCT images. Ultrasonic images are acquired from a linear array of 10 frames per second. The number of pixels of each ultrasonic image is 400×600. OCT images have 500 lines in the rotating direction and 500 pixels in the depth direction. Biological tissue 505 is present between the outer circumference 506 of the puncture needle 202 and the outer periphery of the search needle 203. The cylindrical volume data increases in volume as the search needle 203 moves from a body surface side 503 to the inside 504 of the body. The biological tissue 505 includes latex vessels, interstitial tissue, lobules, blood vessels, and a lesion. After a desired area is specified from the OCT images acquired using the search needle 203, the insertion angle and stop position of the search needle 203 are determined so that tissue is put into the storage portion.

In step A6, the control unit 101 combines the ultrasonic images and the OCT images. As shown in FIG. 5A, the ultrasonic image shows the positional relationship between the target 403 and the search needle 203. An OCT image 501 and an OCT image 502 perpendicular thereto in the ultrasonic image are extracted from the volume data of the OCT unit. OCT images in the longitudinal direction are acquired every 10 micrometers or 100 micrometers. The penetration depth of the search needle 203 and the position of feature portions of the OCT images in the ultrasonic image are combined. The positional relationship between the biopsy probe 108 and the target 403 is calculated from the data on the penetration depth of the search needle 203 and by aligning the feature portions in the ultrasonic images and the feature portions in the OCT images corresponding thereto. Examples of the feature portions include nodes of blood vessels or latex vessels, the boundary between the lesion 403 and normal tissue 404, and the boundary between different tissues.

In step A7, the puncture state of the search needle 203 is controlled. The relationship between the 3D ultrasonic images acquired in step A2 and the OCT images is deduced from the relationship between the ultrasonic images and the OCT images acquired in step A6. Here, the penetration angle of the biopsy probe 108 and the penetration distance thereof to the next step are determined on the basis of the positional relationship between the biopsy probe 108 and the target 403. Since the breast is deformed as the search needle 203 is inserted, an error occurs in the path. Thus, it is necessary to move the search needle 203 forwards while the path is updated successively.

In step A8, it is determined whether the search needle 203 has reached the target 403. Criterion for determination whether the search needle 203 has reached a desired position uses ultrasonic images. If it is determined that the search needle 203 has reached the target 403, the process goes to step A9. If it is determined that the search needle 203 has not reached the target 403, the process returns to step A4. Here, it can be determined using OCT images whether the search needle 203 has passed through a path in which desired tissue is included. There may be a special case in which it is determined using ultrasonic images that the search needle 203 has reached the target 403, but desired tissue is not included. In this case, the details are displayed, and the measurement is stopped without returning to step A4, and an operator's decision is waited for. If the operator decides to stop the measurement, the search needle 203 is drawn out.

In step A9, a puncture position is set. The puncture position is set to the target 403 in an ultrasonic image and is checked after being enlarged in an OCT image. The OCT unit 102 images changes in refractive index. The resolution is about 10 micrometers, which is close to that of an optical microscopic image. Therefore, calcified minute peripheral tissue having different refractive indices is displayed in the image.

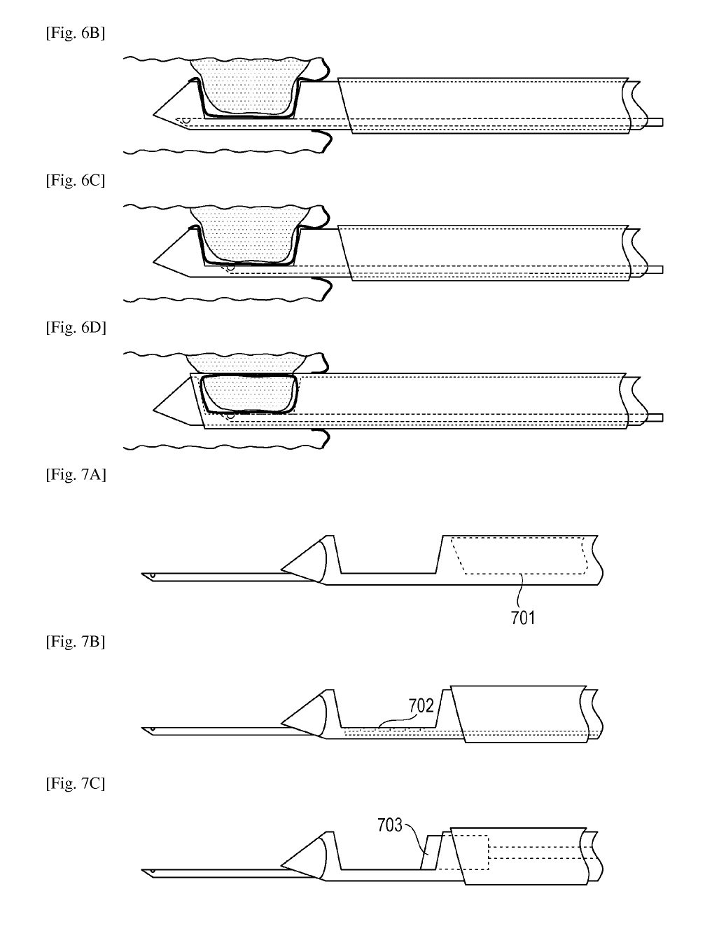

FIGS. 6A to 6D are schematic diagrams in the case where tissue in the periphery of a lesion 602 is to be collected. FIG. 6A shows a state in which the search needle 203 projects from the puncture needle 202, and the lesion 602 is located above the search needle 203. In this case, the search needle 203 may be set in the lesion 602. In addition to the present optical tomographic image during image acquisition, an optical tomographic image before the puncture needle 202 is inserted may be displayed on the display device 106.

In step A10, tissue is collected. FIG. 6B shows the insertion position of the storage portion 204. The puncture needle 202 is slid using the search needle 203 as a guide. The use of the search needle 203 as a guide prevents the puncture needle 202 from deviating from the observation position. In this state, the search needle 203 is moved so that the distal end comes to a position along the storage portion 204. FIG. 6C shows a state in which an OCT image of the lesion 602 is acquired using the search needle 203. If desired tissue is stored in the storage portion 204, the biological tissue is cut off using the external cylindrical cutter 207, as shown in FIG. 6D. After the tissue is cut off, the tissue in the storage portion may be observed by OCT imaging. This can be used for marking in pathological diagnosis. For biopsy of another portion in the same path, the puncture needle 202 is drawn, with the search needle 203 left. After the tissue is collected from the storage portion 204, the puncture needle 202 may be inserted again.

In step A11, the puncture is finished. The puncture needle 202 is drawn out of the living organism. To finish the biopsy, necessary treatment, such as hemostasis, is performed.

Controlling the puncture state of the biopsy probe 108 while comparing ultrasonic images and OCT images allows the target 403 to be approached quickly. The use of OCT images allows determination whether desired tissue is present in the target 403. Complementing the disadvantage of ultrasonic images can increase the accuracy of biopsy.

Although this embodiment uses OCT images, other optical images, such as light scattering images, fluorescence images, Raman images, or infrared images, may be acquired from a living organism. If images of higher quality can be acquired in combination with a contrast medium, the contrast medium may be of course used. Alternatively, optical images may be acquired by a method for emitting light from a search needle in a living organism and acquiring an optical acoustic signal with an ultrasonic probe outside the living organism. Of course, a combination of such optical images may be used.

Furthermore, a treatment in which light different from that for observation may be emitted from a search needle in a living organism, and biological tissue typified by cancer cells is burned may be performed.

Furthermore, the search needle 203 may be inserted while deformation of the breast due to the puncture needle 202 is being simulated. A sufficient diagnosis using optical images would eliminate the use of a tissue collection mechanism.

### Second Embodiment

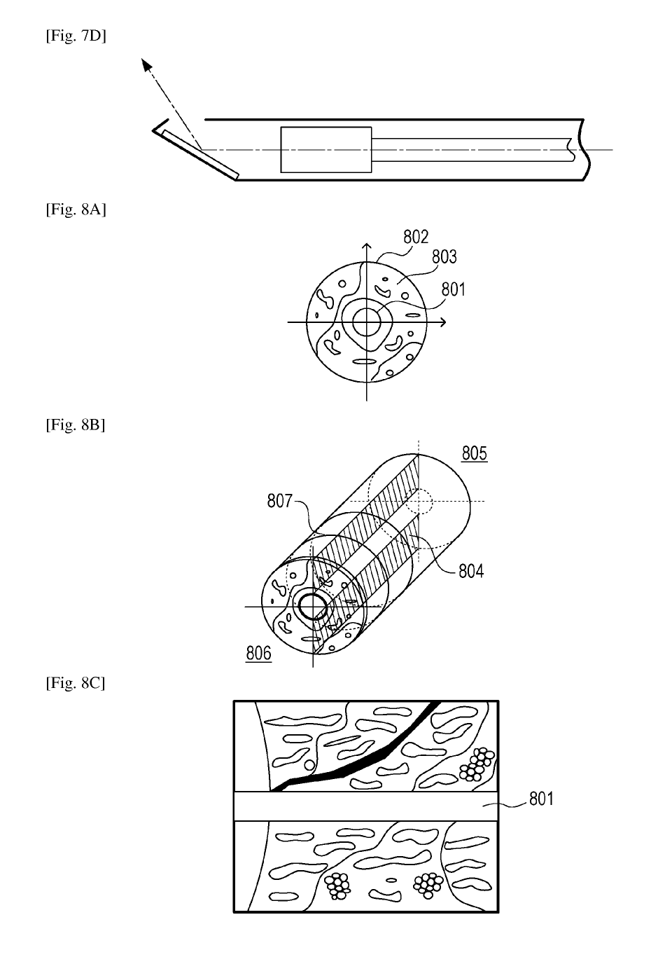

FIGS. 7A to 7D show other puncture needles. FIG. 7A shows a puncture needle having an internal cutter 701. Since the tissue cutting portion is disposed inside, friction with biological tissue can be eliminated, as compared with that disposed outside, thus allowing the tissue cutting portion to be moved smoothly. FIG. 7B shows a puncture needle having an air hole 702 in the bottom of the storage portion 204 of the puncture needle 202. The air hole 702 allows control of air pressure, thus allowing biological tissue to be taken in and out of the storage portion 204 smoothly. Decreasing the air pressure when biological tissue is not sufficiently in close contact will bring the biological tissue into close contact. In contrast, if the biological tissue is not desired tissue, so that it is to be taken out of the storage portion 204, the biological tissue is pushed out by increasing the pressure. FIG. 7C shows a puncture needle in which the storage portion 204 can be changed in size. Adjusting the storage portion 204 to the target biological tissue with an adjusting mechanism 703 allows a necessary minimum portion to be cut off. FIG. 7D shows a configuration in which a mirror is attached at an acute angle smaller than 45 degrees to emit a laser beam far from the distal end of the search needle 203. This allows observation of tissue ahead of the search needle 203 and is thus suitable for control for changing the penetration path.

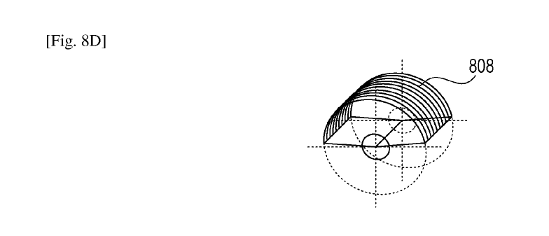

FIGS. 8A to 8D show the details of OCT imaging using the search needle 203. FIG. 8A is a schematic diagram of an OCT image acquired by rotating the search needle 203 one turn. The wavelength of a laser beam for OCT imaging is set to a central wavelength of 1.3 micrometers, at which the laser beam can easily enter a living organism. The light penetration depth is within 3 mm, although it depends on the tissue. One OCT image is constituted by 500 lines in the rotating direction and 500 pixels in the depth direction. In the case of 100 revolutions per 1 millimeter, a longitudinal image with a pitch of 10 micrometers is acquired. Image data corresponds to body tissue 803 between the outer periphery 801 of the search needle 203 and the outermost circumference 802 of the puncture needle 202. Internal tissue 803 includes latex vessels, interstitial tissue, lobules, blood vessels, and a lesion.

FIG. 8B shows volume data acquired in the moving direction. This shows a case in which the search needle 203 enters from a body surface side 805 to the inside 806 of the body cylindrically while performing OCT imaging. FIG. 8C shows an OCT image 804 of the volume data, which is parallel to the longitudinal direction of the search needle 203. The OCT image 804 has 400×600 pixels, which are fixed in the vertical direction, each having a resolution of 10 micrometers, thus allowing an area of 4 mm to be displayed. The resolution in the lateral direction can be changed to 10 micrometers, 20 micrometers, 50 micrometers, or 100 micrometers, thus allowing an area of 6 mm, 12 mm, 30 mm, or 60 mm to be displayed, respectively. If the penetration path is to be viewed, a lower resolution is selected. If the state of tissue at the distal end of the search needle 203 is to be checked, the lowest resolution is selected. This allows both an image of tissue and an image of the outer periphery 801 of the search needle 203 to be acquired, thus making it easy to check the state of tissue to be collected as an image.

Upon confirmation that the search needle 203 has entered the target (lesion) 403 using the ultrasonic image, the linear movement of the search needle 203 is stopped. FIG. 8D shows an observation area 808 at excising positions 807. An OCT image acquired using the search needle 203 is checked, and the excising positions 807 are set. After the setting, the puncture needle 202 is inserted using the search needle 203 as a guide.

### Third Embodiment

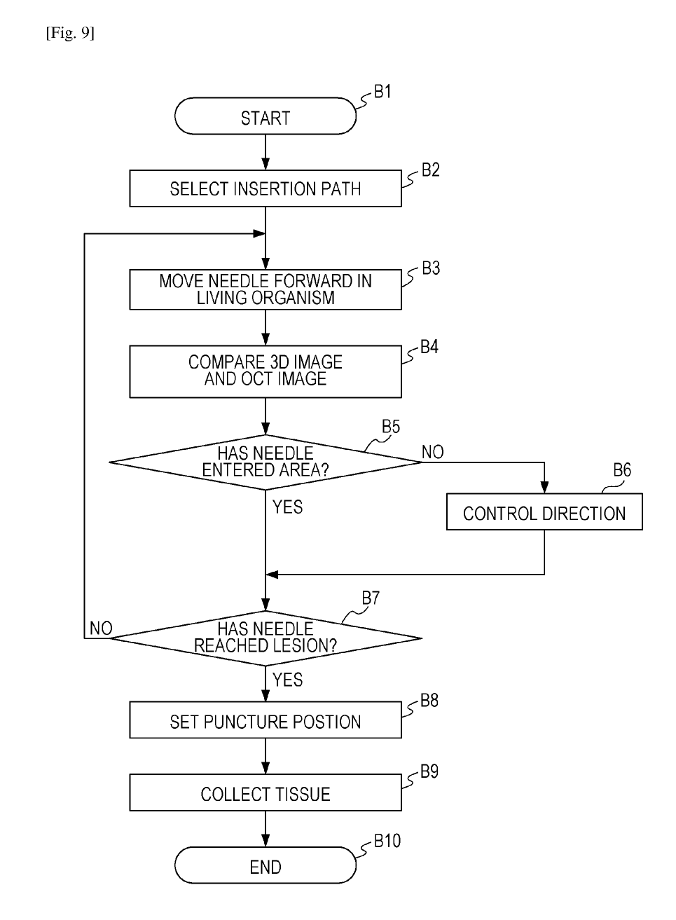

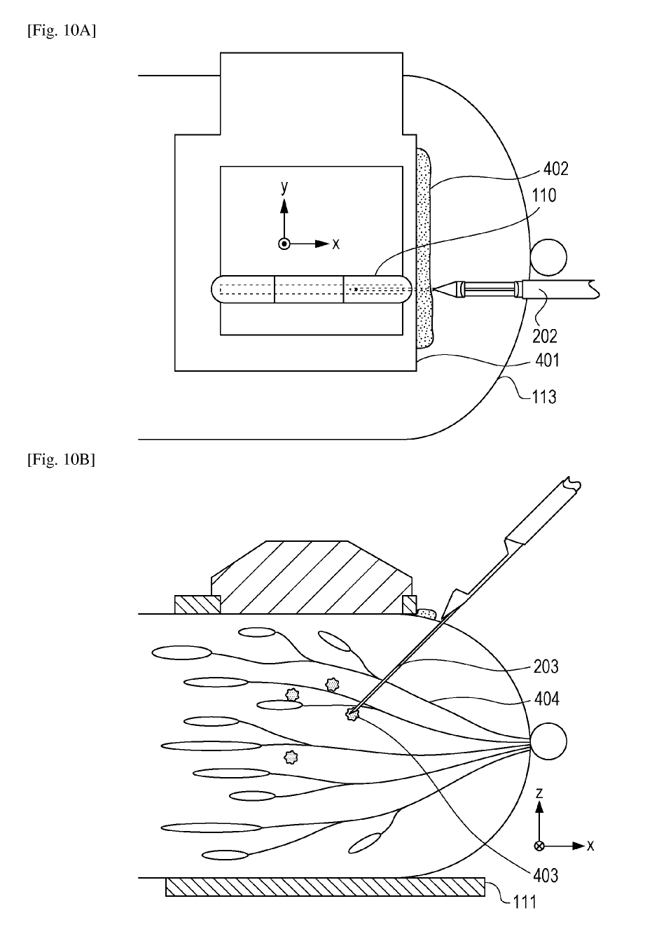

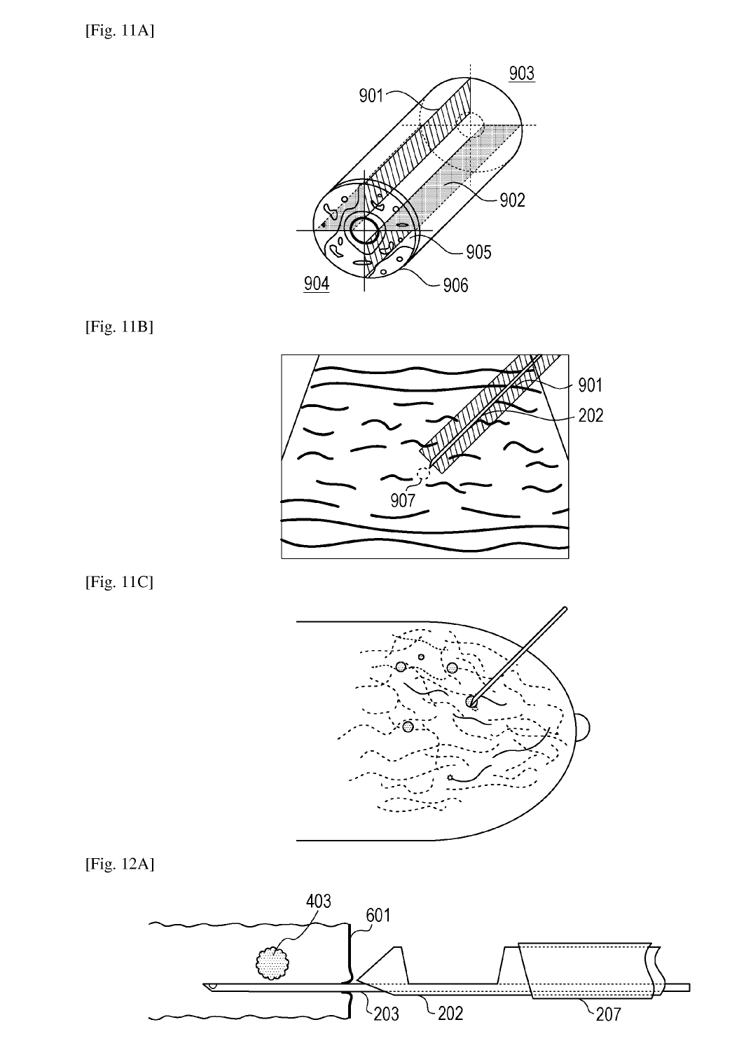

A puncture process controlled by the control unit 101 will be described using a flowchart in FIG. 9. For ease of explanation, FIGS. 10A and 10B show the relationship among the breast 113, the ultrasonic probe 110, and the search needle 203. FIG. 10A is a top view, and FIG. 10B is a side view. Assume that a linear array of the ultrasonic probe 110 is disposed parallel to an x-y plane. FIGS. 11A to 11C show schematic diagrams illustrating the relationship among OCT images, an ultrasonic image, and a mammography image. FIG. 11A illustrates OCT volume data, FIG. 11B illustrates an ultrasonic image, and FIG. 11C illustrates a mammography image. Shifts to the individual steps are performed by the operation of the operator.

In step B1, puncture is started. The breast 113 is subjected to disinfection and anesthesia as appropriate. Although measurement is performed in a standing position, another position, such as a sitting position or a supine position using a chair or a bed, is possible. The breast 113 is sandwiched between the pressure plate 401 and the breast examining table 111. Jell 402 is applied to the breast 113 for acoustic impedance matching.

In step B2, the penetration path is selected. First, a 3D ultrasonic image is acquired by the ultrasonic probe 110. The ultrasonic unit 104 performs electronic scanning in the x-direction and mechanical scanning in the y-direction. In addition, mapping of a 3D blood vessel structure may be performed by Doppler imaging. Furthermore, elastic mapping of tissue may be performed in measurement using elastography or the like. Combination use of them will increase information for alignment. Next, the 3D ultrasonic image acquired above and a mammography image acquired in advance are compared for matching. The mammography apparatus used is preferably a digital mammography apparatus capable of acquiring a 3D image, such as a tomosynthesis apparatus. The matching of images is performed by detecting feature portions (large lesions other than latex vessels, interstitial tissue, lobules, blood vessels, and calcified minute tissue) from the individual images, processing the images so that the positions match, and combining them. The lesion 403 specified by mammography is superposed on the ultrasonic image and is displayed as a virtual lesion 907. The position of the puncture needle 202 is determined on the basis of a penetration path selected depending on the position of the virtual lesion 907. The ultrasonic probe 110 is fixed at a position where the puncture needle 202 and the lesion 403 are in the same plane.

In step B3, the search needle 203 is inserted into the breast 113, and the puncture state is controlled. The search needle 203 is extended from the puncture needle 202, and OCT imaging is performed while the search needle 203 is inserted into the tissue. Since the search needle 203 is rotated in a direction perpendicular to the longitudinal direction, a cylindrical 3D OCT image can be acquired as it moves forwards. Straight movement is controlled independently from rotation; 1 mm per second, or, for example, 0.5 mm or 2 mm, according to circumstances. For rotation, the search needle 203 is rotated at 10 revolutions per millimeter or 100 revolutions per millimeter to acquire an image. Control of straight movement and rotation can be selected as appropriate; for example, every 100 meters for 10 revolutions per millimeter and continuously for 100 revolutions per millimeter. Image acquisition may be increased as the search needle 203 approaches the target lesion 403. Ultrasonic images, if having a resolution of about 400 micrometers, are aligned with OCT images acquired every 100 micrometers.

In step B4, OCT images and ultrasonic images are compared. First, the OCT images have 500 lines in the rotating direction and 500 pixels in the depth direction. Body tissue 905 is present between the outer circumference 906 of the puncture needle 202 and the outer periphery of the search needle 203. The body tissue 905 includes latex vessel, interstitial tissue, lobules, blood vessels, and a lesion. As shown in FIG. 11A, cylindrical volume data is generated as the search needle 203 moves from a body surface side 903 to the inside 904 of the body. An OCT image 901 and an OCT image 202 perpendicular thereto in the ultrasonic image are extracted from the volume data. OCT images in the longitudinal direction are acquired every 10 micrometers or 100 micrometers.

The ultrasonic images have a frame rate of 10 frames per second 400×600 pixels. As shown in FIG. 11B, the ultrasonic images show insertion of the search needle 203. The ultrasonic images and OCT images corresponding thereto are superimposed on the basis of comparison between data on the OCT images and data on the ultrasonic images and the moving distance of the search needle 203. The comparison between data on the OCT images and the ultrasonic images are made on the body tissue 905, which are feature portions in the individual images. Furthermore, the position of the search needle 203 in the mammography image is estimated from the position of the search needle 203 in the ultrasonic images. FIG. 11C is a mammography image on which a planar image 901 including the estimated search needle 203 is superimposed. The operator checks the lesion 403 and the distal end of the search needle 203.

In step B5, it is determined whether the search needle 203 has entered an area in which the lesion 403 can be reached. The destination of the search needle 203 moved is estimated on the basis of the position of the search needle 203 in the mammography image acquired in step B4. If the search needle 203 cannot reach the lesion 403, the process goes to step B6, and if can reach the lesion 403, the process goes to step B7.

In step B6, the directions of the search needle 203 and the ultrasonic probe 110 are controlled. The direction of the search needle 203 is controlled by controlling the direction of the biopsy probe 108 using the biopsy probe arm 112. If the breast 113 is deformed as the search needle 203 is inserted, and the lesion 403 and the search needle 203 come out of the ultrasonic image, ultrasonic probe 110 is also controlled using the ultrasonic probe arm 109. Whether they come out of the ultrasonic image is made by comparing an OCT image 902 perpendicular to the OCT image 901 described above and a corresponding portion in the 3D ultrasonic image. After the directional control, the process goes to step B7.

In step B7, it is determined whether the search needle 203 has reached the lesion 403. Criterion for determination whether the search needle 203 has reached a desired position uses ultrasonic images. If it is determined that the search needle 203 has reached the lesion 403, the process goes to step B8. If it is determined that the search needle 203 has not reached the lesion 403, the process returns to step B3. Here, it can be determined using OCT images whether the search needle 203 has passed through a path in which desired tissue is included. There may be a special case in which it is determined using ultrasonic images that the search needle 203 has reached the lesion 403, but desired tissue is not included. In this case, the details are displayed, and the measurement is stopped without returning to step B3, and an operator's decision is waited for.

In step B8, a puncture position is set. The puncture position is checked by the operator using an OCT image in which the distal end of the search needle 203 and tissue in the vicinity of the lesion 403 (the vicinity of the target 403) are displayed on the same screen. The OCT unit 102 images changes in refractive index. The resolution is about 10 micrometers, which is close to that of an optical microscopic image. Therefore, calcified minute peripheral tissue having different refractive indices is displayed in the image.

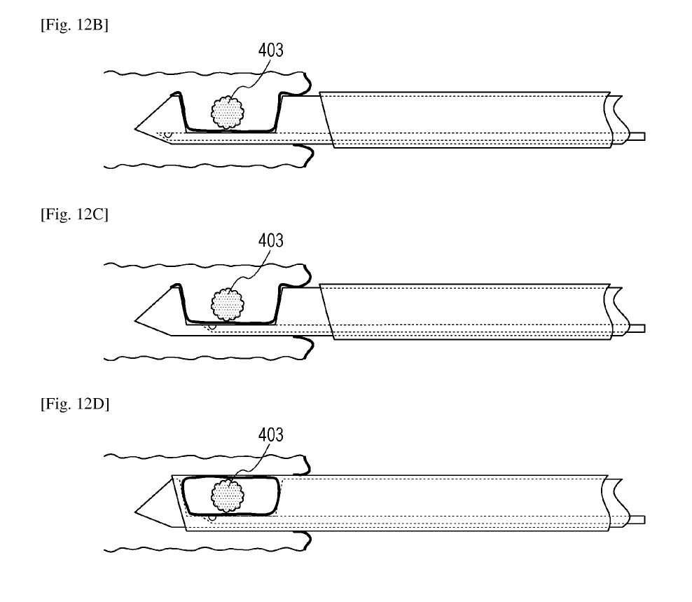

In step B9, tissue is collected. FIGS. 12A to 12D are schematic diagrams illustrating the state of collection of tissue. FIG. 12A illustrates the positional relationship between the search needle 203 and the lesion 403 in a living organism 601. Here, calcified lesion is to be collected. The position of the search needle 203 is set so the lesion 403 is stored in the storage portion 204. As shown in FIG. 12B, the puncture needle 202 is inserted using the search needle 203 as a guide. The use of the search needle 203 as a guide prevents the puncture needle 202 from deviating from the set position. In this state, the position and angle of the storage portion 204 relative to the search needle 203 are determined. FIG. 12C shows a state in which an OCT image of the lesion 403 is acquired by the search needle 203. In addition to the present optical tomographic image during image acquisition, an OTC image before the puncture needle 202 is inserted may be displayed on the display device 106. After confirming that the position of the storage portion 204 is correct, the tissue is cut off with the external cylindrical cutter 207. FIG. 12D illustrates a state in which tissue is cut. After the excised tissue is stored in the storage portion 204, an OCT image may be acquired in the external cylindrical cutter 207. This can be used for marking in pathological diagnosis. This can be used for marking in pathological diagnosis. For biopsy of another portion in the same path, the puncture needle 202 is drawn, with the search needle 203 left. After the tissue is collected from the storage portion 204, the puncture needle 202 may be inserted again.

In step B10, the puncture needle 202 drawn out of the breast 113, and the puncture is finished. To finish the biopsy after collecting the tissue, necessary treatment, such as hemostasis, is performed.

Thus, the position of the lesion 403, which cannot be detected in the ultrasonic images, can be set in the ultrasonic images by aligning the mammography image and the ultrasonic images. Inserting the search needle 203 while comparing the ultrasonic images and the OCT images allows efficient approach to the lesion 403. Furthermore, it can be checked whether desired tissue is present in the lesion 403 using OCT images.

Although this embodiment uses OCT, other optical images, such as light scattering images fluorescence images, Raman images, or infrared images, may be acquired from a living organism. If images that are more accurate can be acquired in combination with a contrast medium, the contrast medium may be of course used. A combination of such optical images may be used. Examples of the 3D-image acquisition unit for acquiring images from the outside of a living organism include not only the mammography unit but also an X-ray CT scanner, a nuclear magnetic resonator, and a positron emission tomography apparatus. Ultrasonic images themselves may be used. Lesion detected by the above modalities can be punctured under ultrasonic guidance.

In contrast, although this embodiment uses the ultrasonic image acquisition unit, it is not absolutely necessary if the optical images have a sufficient invasion depth, and the correlation between the optical images and a 3D image is easy. The search needle 203 may be inserted while deformation of the breast 113 due to the puncture needle 202 is being simulated. A sufficient diagnosis using optical images would eliminate the use of a tissue collection mechanism. Alternatively, optical images may be acquired by emitting light from a search needle in a living organism and acquiring an optical acoustic signal with an ultrasonic probe in the surrounding. Furthermore, a treatment in which light different from that for observation may be emitted from a search needle, and biological tissue typified by cancer cells is burned may be performed.

Although the above embodiments use the breast by way of example, another organ, such as a liver or a prostate, may be used.

Aligning ultrasonic images and OCT images allows the position of the puncture needle 202 to be confirmed and to reach the target. Furthermore, OCT images can be acquired by the search needle 203, so that desired tissue around the puncture needle 202 can be observed at an optical microscopic level.

Furthermore, since the state of target tissue can be checked by OCT imaging, and the position of the puncture needle 202 can be adjusted using the search needle 203 as a guide, unnecessary biopsy can be reduced, and the success rate of biopsy can be increased.

The above embodiments can perform puncture by introducing the puncture needle 202 to the vicinity of a lesion using image data obtained by past mammography and confirming tissue in the vicinity of the search needle 203 using the OCT unit 102. This allows ultrasonic-guided biopsy also for a calcified minute lesion that cannot be detected by an ultrasonic unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-125069, filed Jun. 13, 2013, No. 2013-125070, filed Jun. 13, 2013, and No. 2013-125071, filed Jun. 13, 2013, which are hereby incorporated by reference herein in their entirety.

### REFERENCE SIGNS LIST

110 ultrasonic probe

104 ultrasonic unit

108 biopsy probe

102 optical-image acquisition unit

101 control unit

403 target

601 biological tissue

208 exit window

203 search needle

204 storage portion

202 puncture needle

113 subject

1. A biopsy support apparatus comprising:
an ultrasonic unit configured to generate an ultrasonic image on the basis of a signal from an ultrasonic probe in contact with a living organism;
a biopsy probe configured to be inserted in the living organism; and
an optical-image acquisition unit configured to generate the an optical image on the basis of a signal from the biopsy probe inserted in the living organism
wherein the biopsy probe comprises:
a search needle configured to output a signal for generating the optical image; and
a puncture needle including a storage portion for storing the biological tissue of a target collected, the puncture needle being slidable using the search needle as a guide, and
wherein the puncture needle includes a cutting portion configured to cut off the biological tissue stored in the storage portion.

2. The biopsy support apparatus according to claim 1, wherein the feature portion is a node of blood vessels or latex vessels, the boundary between a lesion and normal tissue, or the boundary between different tissues.

3. A method for supporting biopsy, the method comprising the steps of:
generating an ultrasonic image on the basis of a signal from an ultrasonic probe in contact with a living organism;
generating an optical image on the basis of a signal from a biopsy probe inserted in the living organism;
setting a scanning direction of the ultrasonic probe and a moving direction of the biopsy probe in substantial coplanar manner;
calculating a positional relationship between the biopsy probe and the a target by aligning a feature portion in the ultrasonic image and a feature portion corresponding thereto in the optical image; and
controlling a puncture state of the biopsy probe on the basis of the positional relationship,
wherein the biopsy probe comprises:
a search needle configured to output a signal for generating the optical image; and
a puncture needle including a storage portion for storing the biological tissue of the target collected, the puncture needle being slidable using the search needle as a guide, and
wherein the puncture needle includes a cutting portion configured to cut off the biological tissue stored in the storage portion.

4. The biopsy support apparatus according to claim 1,
wherein optical images are acquired from the optical-image acquisition unit in a first state in which the exit window projects from the storage portion and in a second state in which the exit window is located along the storage portion.

5. The biopsy support apparatus according to claim 4, wherein the optical image in the second state includes an image of the biological tissue and an image of the search needle itself.

6. The biopsy support apparatus according to claim 4, wherein the air pressure of the storage portion can be controlled.

7. The biopsy support apparatus according to claim 1, further comprising a biopsy probe arm configured to control the operation of the biopsy probe.

8. The biopsy support apparatus according to claim 1, wherein the optical image is an OCT image.

9. A method for controlling a biopsy support apparatus, wherein the biopsy support apparatus comprises:
a search needle including an optical device for acquiring an optical image of biological tissue and an exit window; and
a puncture needle including a storage portion configured to store the part of the biological tissue collected, the puncture needle being slidable using the search needle as a guide,
wherein the puncture needle includes a cutting portion configured to cut off the biological tissue stored in the storage portion, and
wherein optical images are acquired in a first state in which the exit window projects from the storage portion and in a second state in which the exit window is located along the storage portion.

10. The biopsy support apparatus according to claim 1, further comprising:
a biopsy probe configured to be inserted into a subject;
an optical-image acquisition unit configured to generate an optical image of the subject by inserting the biopsy probe into the subject; and
a control unit configured to control the optical-image acquisition unit and the puncture state of the biopsy probe, wherein the control unit acquires data on a 3D image of the subject acquired by a 3D-image acquisition unit in advance; and determines the penetration path of the biopsy probe on the basis of the data on the 3D image.

11. The biopsy support apparatus according to claim 11, wherein the biopsy probe being inserted into the subject and tissue in the vicinity of the target are displayed on the same screen of the optical image.

12. The biopsy support apparatus according to claim 11, further comprising
an ultrasonic unit configured to generate an ultrasonic image by bringing an ultrasonic probe into contact with the subject,
wherein the control unit causes the target to be displayed in the ultrasonic image by using the data on the 3D image.

13. The biopsy support apparatus according to claim 13, wherein the control unit causes the target to be displayed in the ultrasonic image by detecting a feature portion of the 3D image and a feature portion of the ultrasonic image and aligning the feature portions.

14. The biopsy support apparatus according to claim 11, wherein the 3D-image acquisition unit is a mammography unit.

15. The biopsy support apparatus according to claim 11, wherein the optical-image acquisition unit is an OCT unit.

16. The biopsy support apparatus according to claim 1, further comprising:
a control unit configured to control the ultrasonic unit and the optical-image acquisition unit,
wherein the control unit sets the scanning direction of the ultrasonic probe and the moving direction of the biopsy probe in substantially the same plane; calculates the positional relationship between the biopsy probe and the target by aligning a feature portion in the ultrasonic image and a feature portion corresponding thereto in the optical image; and controls the puncture state of the biopsy probe on the basis of the positional relationship.

17. (canceled)

18. A method for puncturing a target in a subject, the method comprising the steps of:
generating an optical image of the subject by inserting a biopsy probe into a subject;
controlling the puncture state of the biopsy probe; acquiring data on a 3D image of the subject acquired by a 3D-image acquisition unit in advance; and
determining the penetration path of the biopsy probe on the basis of the data on the 3D image,
wherein the biopsy probe comprises:
a search needle configured to output a signal for generating the optical image; and
a puncture needle including a storage portion for storing the biological tissue of the target collected, the puncture needle being slidable using the search needle as a guide, and
wherein the puncture needle includes a cutting portion configured to cut off the biological tissue stored in the storage portion.

19. A biopsy support apparatus, the apparatus comprising:
an ultrasonic unit configured to generate the ultrasonic image on the basis of a signal from an ultrasonic probe in contact with the living organism;
a biopsy probe configured to be inserted in the living organism;
an optical-image acquisition unit configured to generate the optical image on the basis of a signal from the biopsy probe inserted in the living organism; and
a control unit configured to control the ultrasonic unit and the optical-image acquisition unit,
wherein the control unit:
sets the scanning direction of the ultrasonic probe and the moving direction of the biopsy probe in substantially the same plane;
calculates the positional relationship between the biopsy probe and a target by aligning a feature portion in the ultrasonic image and the feature portion corresponding thereto in the optical image; and
controls the puncture state of the biopsy probe on the basis of the positional relationship,
wherein the feature portion is a node of blood vessels or latex vessels, the boundary between a lesion and normal tissue, or the boundary between different tissues.

20. A biopsy support apparatus, the apparatus comprising:
an ultrasonic unit configured to generate the ultrasonic image on the basis of a signal from an ultrasonic probe in contact with the living organism;
a biopsy probe configured to be inserted in the living organism;
an optical-image acquisition unit configured to generate the optical image on the basis of a signal from the biopsy probe inserted in the living organism;
a control unit configured to control the ultrasonic unit and the optical-image acquisition unit; and
a biopsy probe arm configured to control the operation of the biopsy probe,
wherein the control unit:
sets the scanning direction of the ultrasonic probe and the moving direction of the biopsy probe in substantially the same plane;
calculates the positional relationship between the biopsy probe and a target by aligning a feature portion in the ultrasonic image and a feature portion corresponding thereto in the optical image; and
controls the puncture state of the biopsy probe on the basis of the positional relationship.
